(19) United States
(12) Patent Application Publication
(10) Pub. No.: US 2015/0065857 A1
(43) Pub. Date: Mar. 5, 2015

(54) Determining a Respiratory Rate of a Living Being

(71) Applicants: Stephan Biber, Erlangen (DE); Michael Wiehl, Ochsenfurt (DE)

(72) Inventors: Stephan Biber, Erlangen (DE); Michael Wiehl, Ochsenfurt (DE)

(21) Appl. No.: 14/474,136

(22) Filed: Aug. 30, 2014

(30) Foreign Application Priority Data
Aug. 30, 2013 (DE) 102013217341.5
Jun. 27, 2014 (DE) 102014212468.9

(51) Int. Cl. A61B 5/08; A61B 5/00; A61B 5/055; A61B 5/05

(52) U.S. Cl. CPC A61B 5/0816; A61B 5/0507; A61B 5/725; A61B 5/055
USPC 600/411; 600/407

(57) ABSTRACT

A method for determining a respiratory rate of a living being includes emitting a radio signal using a radio transmitter arranged on the living being. The emitted radio signal is acquired, and the respiratory rate is determined based on the acquired radio signal.

This application claims the benefit of DE 10 2013 217 341.5, filed on Aug. 30, 2013, and DE 10 2014 212 468.9, filed on Jun. 27, 2014, which are hereby incorporated by reference in their entirety.

### BACKGROUND

The present embodiments relate to determining a respiratory rate of a living being.

In certain situations, the respiratory rate of a living being (e.g., of a human) is to be determined. In the case of whole body imaging methods, the respiratory rate may be established during the whole body imaging method.

When using currently available whole body imaging devices, the respiratory rate is known to be determined in direct contact with the patient. A belt is wound around the patient, with the cyclical expansion of the belt being evaluated (e.g., in the case of BioHarness BT by Zephyr Technology). As an alternative to the belt, an air-filled cushion is fastened to the patient. A further known option for determining the respiratory rate is formed by radar methods (e.g., described in Jelen, M., Biebel, E. M., “Multifrequency Sensor for Remote Measurement of Breath and Heartbeat,” Advances in Radio Science 4 (79), pages 79-83, 2006). A weak continuous wave (CW) signal is radiated onto the patient, and the reflected signal is received and evaluated (e.g., continuous wave radar).

Frequency modulated continuous wave (FMCW) radars may be used for directly measuring the heart and respiratory rates. This technique may be used directly in magnetic resonance imaging devices (MRI devices) (see K. Mostov et al., “Medical Applications of Shortwave FM Radar,” Remote Monitoring of Cardiac and Respiratory Motion, 2010).

Such additional measuring units (e.g., radar measuring units or belts) that are to be buckled on by the patient or are to be fastened manually to the patient are bothersome for the patient or may not be able to be used in a satisfactory manner for reasons of space. Carrying out whole body imaging methods is made significantly more difficult, or these may not be particularly comfortable.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method for determining a respiratory rate of a living being that may be used in a comfortable and space-saving manner, in the case of whole body imaging methods on the living being, is provided. As another example, a respiratory rate determination system, by which the method may be carried out in a suitable manner, is also provided. As yet another example, a magnetic resonance imaging device, by which the method for determining the respiratory rate of the living being may be carried out when carrying out magnetic resonance imaging examinations, is provided.

In the case of the method according to one or more of the present embodiments for determining the respiratory rate of a living being, a radio signal is emitted by a radio transmitter or radio transmission element arranged on the living being, and the emitted radio signal is acquired. Alternatively or additionally, a radio signal is emitted, and the radio signal is acquired by a radio receiver or radio reception element arranged on the living being. According to one or more of the present embodiments, the respiratory rate is determined based on the acquired radio signal.

A radio transmitter may be a device that transmits the radio signal. The radio transmitter may not include possibly present transmission electronics or the like. The radio transmitter initially only includes a radio transmission element in the form of a transmission antenna that is configured for emitting radio signals. Optionally, in addition to the transmission antenna, the term radio transmitter includes an oscillator for feeding the transmission antenna and, for example, an amplifier.

Analogously, a radio receiver may be a device that receives the radio signal. Possibly present evaluation electronics are not necessarily encompassed by the term radio receiver. Rather, the term radio receiver may include only a radio reception element in the form of a reception antenna that is embodied for receiving radio signals. However, optionally, the term radio receiver also includes such evaluation electronics.

Therefore, according to one or more of the present embodiments, a radio transmitter is arranged on the living being. It is not necessary for the living being to wear a bothersome belt. Measuring units (e.g., radar measuring units) that require much space may be dispensed with. The radio transmitter may have a space-saving and wireless design. In this manner, the method may be used without much spatial outlay. For example, the method according to one or more of the present embodiments may be suitably used without further obstacles when carrying out whole body imaging methods with, typically, a reduced available space.

In one embodiment, at least one amplitude and/or at least one frequency and/or at least one phase of the acquired radio signal is used for determining the respiratory rate in the method according to one or more of the present embodiments.

In the method according to one or more of the present embodiments, a radio transmitter that is arranged in the vicinity of the rib cage, the abdomen, or the back of the living being and therefore moves to-and-fro with the rate of respiration, and/or a radio receiver that is arranged in the vicinity of the rib cage or the abdomen or the back of the living being is used. As a result of this, the amplitude of the acquired radio signal varies with the respiratory rate. In one embodiment, the amplitude is used in a time-resolved manner for determining the respiratory rate in the method according to one or more of the present embodiments. The respiratory rate may be determined from the profile of the amplitude over time.

Alternatively or additionally, a change in the radio signal (e.g., over time, in the amplitude and/or in the phase and/or in the frequency, as a result of the change in the transmission properties of the signal path from the transmission element to the reception element) is used in the method according to one or more of the present embodiments for determining the respiratory rate. The radio transmitter may transmit the radio signal with a sufficiently constant frequency and/or amplitude and/or constant phase such that, when the radio signal is acquired, the frequency and/or the amplitude and/or the phase of the acquired radio signal varies with the movement of the radio transmitter. In one embodiment, the frequency of the acquired radio signal is acquired in a time-resolved manner in the method according to one or more of the present embodiments. The respiratory rate may be determined from the time profile of the transmission properties of the acquired radio signal (e.g., of the amplitude and/or of the phase of the radio signal).

Alternatively or additionally to the aforementioned options, the phase of the acquired radio signal may be used. As a result of the periodic movement of the radio transmitter and/or of the radio receiver, there is a change, for example, in the distance between the radio transmitter and a radio acquisition apparatus serving for acquiring the radio signal. As a result of the periodically varying route, the radio signal is acquired with a periodically varying phase. In one embodiment, the phase is acquired in a time-resolved manner in the method according to one or more of the present embodiments. The respiratory rate may be determined from the time profile of the phase of the acquired radio signal.

In an advantageous development, the acquired radio signal is initially subjected to frequency filtering (e.g., low-pass filtering), in the method, and the frequency-filtered radio signal is used for determining the respiratory rate. For example, changes in the radio signal over time due to the movement of the radio transmitter and/or of the radio receiver with the respiratory rate, which may form a low-frequency component of the radio signal, are easily separated from the radio signal and used for determining the respiratory rate. For example, a further frequency range (e.g., a higher frequency range) of the radio signal may be used for transmitting further data. In this development of the method, a frequency range in the range of the typical respiratory rate (e.g., a frequency range of frequencies less than 1 Hz and/or greater than 100 mHz) may be separated out and used for determining the respiratory rate.

The method according to one or more of the present embodiments may be carried out when operating a magnetic resonance imaging device, and the respiratory rate of a living being situated in the magnetic resonance imaging device is determined.

In one embodiment, the method according to one or more of the present embodiments is carried out when operating a magnetic resonance imaging device that includes a reception coil. A message signal received by the reception coil is transmitted to a remaining part of the magnetic resonance imaging device by the radio signal. The remaining part of the magnetic resonance imaging device refers to a part that is not embodied as a local coil. To the extent that this description refers to the term “reception coil”, the reception coil may be synonymous with the term “local coil”. In this development of the method, it follows that the radio transmitter is used not only for establishing the respiratory rate, but the radio transmitter at the same time assumes a further object (e.g., an object already known from the prior art) when operating magnetic resonance imaging devices. Thus, a radio transmitter, by which a message signal that is received by the reception coil is transmitted to at least one remaining part of the magnetic resonance imaging device, may be provided. In this development of the method, a radio transmitter already present for other functions additionally assumes a further function by virtue of the radio transmitter being used for establishing the respiratory rate in accordance with the method according to one or more of the present embodiments.

The respiratory rate determination system according to one or more of the present embodiments is configured to carry out a method as described above for determining the respiratory rate of a living being. The respiratory rate determination system includes a radio transmitter embodied for arrangement on a living being and a radio acquisition apparatus for acquiring a radio signal emitted by the radio transmitter. Alternatively or additionally, the respiratory rate determination system includes a radio transmitter and a radio acquisition apparatus with a radio receiver or radio reception element embodied for arrangement on a living being. The radio acquisition apparatus is, for example, embodied in each case to acquire (e.g., in a time resolved manner) at least one variable (e.g., at least one frequency and/or at least one amplitude and/or at least one phase of the acquired radio signal) and to determine the respiratory rate of the living being depending on the at least one variable. The respiratory rate determination system according to one or more of the present embodiments makes it easy to carry out the method for determining the respiratory rate of a living being, as described above.

In the respiratory rate determination system according to one or more of the present embodiments, the radio acquisition apparatus may include at least one frequency filter (e.g., a low-pass filter). As a result of this, the radio transmitter, as already described above in relation to the method according to one or more of the present embodiments, may be used not only for determining the respiratory rate, but the radio transmitter may simultaneously satisfy further message signal transmission functions (e.g., message signal transmission functions already known in magnetic resonance imaging devices). For example, a respiratory rate-dependent signal component may easily be filtered out using a low-pass filter. In one embodiment, the frequency filter is configured to filter out a signal component with frequencies in the frequency range of the typical respiratory rate (e.g., a frequency range of frequencies less than 1 Hz and/or greater than 100 mHz), such that this signal component may be used for determining the respiratory rate.

In the respiratory rate determination system according to one or more of the present embodiments, the radio acquisition apparatus may include at least one power detector and/or at least one frequency detector and/or at least one phase detector. Using the power detector, the amplitude of the acquired radio signal, for example, may be acquired. Alternatively or additionally, the frequency of the acquired radio signal may easily be acquired by the frequency detector. Alternatively or additionally, the phase of the acquired radio signal may be acquired particularly easily in a corresponding manner using the phase detector.

The magnetic resonance imaging device according to one or more of the present embodiments includes a respiratory rate determination system as described above. In one embodiment, the magnetic resonance imaging device includes a reception coil, where the radio transmitter is signal-connected to the reception coil and configured to emit a message signal received by the reception coil. In one embodiment, the message signal emitted by the radio transmitter simultaneously forms the radio signal.

### BRIEF DESCRIPTION OF THE DRAWINGS

FIG. 1 schematically shows one embodiment of a respiratory rate determination system for determining a respiratory rate of a living being; and

FIG. 2 schematically shows a further embodiment of the respiratory rate determination system for determining the respiratory rate of a living being.

### DETAILED DESCRIPTION

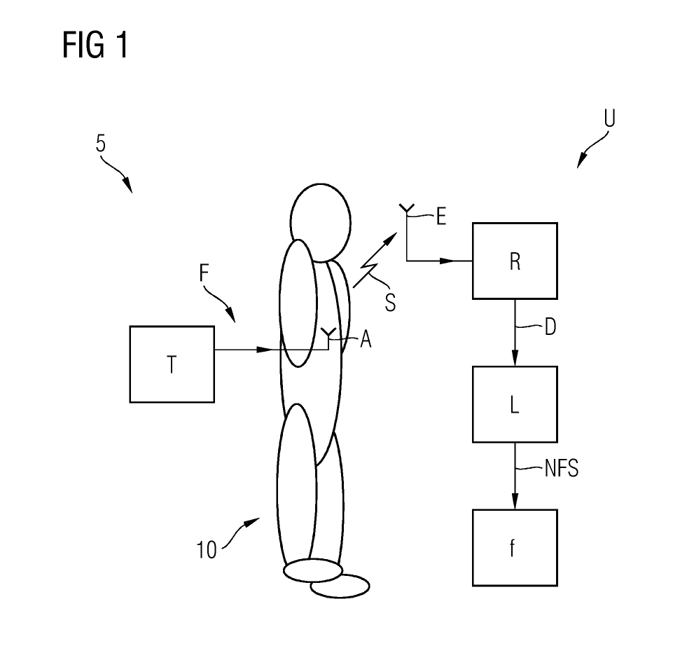

A respiratory rate of a human 10 is determined by a respiratory rate determination system 5 according to one or more of the present embodiments, depicted in FIG. 1. The human 10 wears a radio transmitter F, which includes a transmission antenna A and transmission electronics T for feeding the transmission antenna A. The transmission antenna A is arranged on the human 10 in the vicinity of the abdomen of the human 10 and therefore moves to-and-fro with the rate of respiration. In a manner known, the transmission antenna A transmits a radio signal S including radio data to a reception antenna E. The reception antenna E is arranged in a movement-decoupled manner from the human 10 and the respiratory movement of the human 10, and receives the radio signal S. The reception antenna E is part of a radio acquisition apparatus U that includes reception electronics R, fastened to the reception antenna E, for evaluating the received radio signal S. The radio acquisition apparatus U also includes a low-pass filter L and a frequency detection unit f. The reception electronics R process the received radio signal S and extract the received radio signal S using an amplitude demodulator (not explicitly depicted in FIG. 1) and using a frequency demodulator (not explicitly depicted in FIG. 1). After these demodulations, a demodulated signal D that includes a radiofrequency signal component including the radio data and a low-frequency signal component NFS is available in each case. The low-frequency signal component NFS is caused by the movement of the radio transmitter F as a result of the respiratory movement of the human 10. In this case, low-frequency denotes a range of frequencies greater than 100 mHz and less than 1 Hz. The demodulated signal D is subsequently transmitted from the reception electronics R to the low-pass filter L, which is signal-connected to the reception electronics for this purpose. The low-pass filter L separates the low-frequency signal component NFS from the demodulated signal D and therefore merely outputs frequencies in the range of typical respiratory rates. This low-frequency signal component NFS is transmitted to a frequency detection unit f that detects the respiratory rate from the low-frequency signal component NFS. The frequency detection unit reads the frequency using a digital Fourier transform and a subsequent search for a maximum or using a period duration measurement.

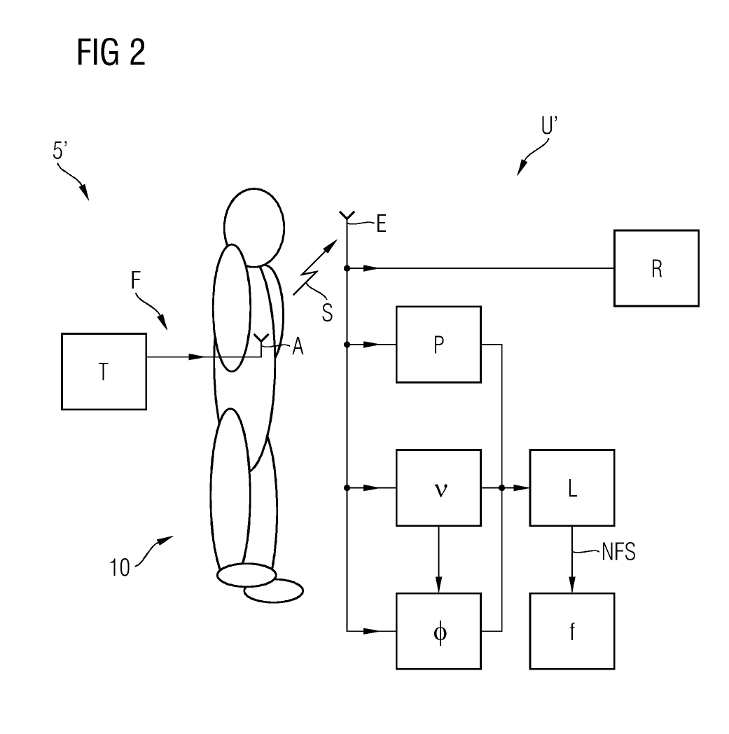

The respiratory rate determination system 5′ depicted in FIG. 2 corresponds to the respiratory rate determination system 5 depicted in FIG. 1. Deviating from the respiratory rate determination system 5 depicted in FIG. 1, the amplitude, the phase, and the frequency in the respiratory rate determination system 5′ depicted in FIG. 2 are extracted directly from the radio signal S received by the reception antenna E. The radio acquisition apparatus U′ includes a power detector P, a frequency detector v and a phase detector φ. The power detector P, the frequency detector v and the phase detector φ are directly signal-connected to the reception antenna E in each case. In the depicted exemplary embodiment, the power detector P, the frequency detector v and the phase detector φ are line-connected by electrical lines. By contrast, the reception electronics R are connected to the reception antenna E independently of the power detector P, the frequency detector v and the phase detector φ. Analogously to the exemplary embodiment depicted in FIG. 1, the power detector P, the frequency detector v and the phase detector φ in each case transmit the detected signals thereof to a low-pass filter, which transmits a low-frequency signal component NFS to a frequency detection unit f.

Using the exemplary embodiment depicted in FIG. 2, the method according to one or more of the present embodiments may be carried out independently of the type of modulation for modulating radio data of the radio signals, to the extent that the radio data does not occupy the frequency range of the respiration. Therefore, radio transmitter F and radio acquisition apparatus U′ may also apply digital methods in further exemplary embodiments not specially depicted here.

The depicted exemplary embodiments for respiratory rate determination systems 5, 5′ are configured for carrying out the method according to one or more of the present embodiments. The respiratory rate determination systems 5, 5′ are part of a magnetic resonance imaging device (not explicitly depicted here). The radio transmitter F is embodied to transmit a message signal from a reception coil (not explicitly depicted here) of the magnetic resonance imaging device to a message processing device.

In not specially depicted exemplary embodiments, which incidentally correspond to the exemplary embodiments explained above, the respiratory rate determination system is configured for determining the respiratory rate not of a human 10 but of an animal, and the above-described method is carried out for determining the respiratory rate of the animal.

In further, not specially depicted exemplary embodiments, the roles of the transmission antenna A of the radio transmitter F and of reception antenna E of the radio acquisition apparatus U, U′ are interchanged (e.g., the reception antenna E is arranged on the human 10 in the vicinity of his abdomen, where the transmission antenna A is arranged in a movement-decoupled manner from the human 10).

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining a respiratory rate of a living being, the method comprising:
emitting, by a radio transmitter or a radio transmitter element, a radio signal arranged on the living being and acquiring the emitted radio signal, emitting the radio signal and acquiring, by a radio receiver or a radio reception element arranged on the living being, the emitted radio signal, or a combination thereof; and
determining the respiratory rate based on the acquired radio signal.

2. The method of claim 1, wherein determining the respiratory rate comprises using at least one amplitude of the acquired radio signal, at least one frequency of the acquired radio signal, at least one phase of the acquired radio signal, or any combination thereof.

3. The method of claim 1, wherein determining the respiratory rate comprises using a change in the radio signal.

4. The method of claim 3, wherein using the change in the radio signal comprises using a change in an amplitude of the acquired radio signal, a change in a frequency of the acquired radio signal, a change in a phase of the acquired radio signal, or any combination thereof.

5. The method of claim 1, wherein the acquired radio signal is initially subjected to frequency filtering, and the frequency-filtered radio signal is used for determining the respiratory rate.

6. The method of claim 5, wherein the frequency filtering is low-pass filtering.

7. The method of claim 1, wherein the method is carried out when operating a magnetic resonance imaging device, and
wherein determining the respiratory rate comprises determining the respiratory rate of the living being, which is situated in/on the magnetic resonance imaging device.

8. The method of claim 1, wherein the method is carried out when operating a magnetic resonance imaging device with a reception coil, and
wherein the method further comprises transmitting a message signal received by the reception coil to a remaining part of the magnetic resonance imaging device by the radio signal.

9. A respiratory rate determination system for determining a respiratory rate of a living being, the respiratory rate determination system comprising:
a radio transmitter arrangeable on the living being; and
a radio acquisition apparatus configured for acquiring a radio signal emitted by the radio transmitter, a radio receiver or a radio reception element arrangeable on the living being, or a combination thereof,
wherein the radio acquisition apparatus is configured to acquire at least one variable of the acquired radio signal and to determine the respiratory rate of the living being depending on the at least one variable.

10. The respiratory rate determination system of claim 9, wherein the at least one variable comprises at least one frequency, at least one amplitude, at least one phase, or any combination thereof.

11. The respiratory rate determination system of claim 9, wherein the radio acquisition apparatus comprises at least one frequency filter.

12. The respiratory rate determination system of claim 11, wherein the at least one frequency filter comprises a low-pass filter.

13. The respiratory rate determination system of claim 9, wherein the radio acquisition apparatus comprises at least one power detector, at least one frequency detector, at least one phase detector, or any combination thereof.

14. A magnetic resonance imaging device comprising:
a respiratory rate determination system for determining a respiratory rate of a living being, the respiratory rate determination system comprising:
a radio transmitter arrangeable on the living being; and
a radio acquisition apparatus configured for acquiring a radio signal emitted by the radio transmitter, a radio receiver or a radio reception element arrangeable on the living being, or a combination thereof,
wherein the radio acquisition apparatus is configured to acquire at least one variable of the acquired radio signal and to determine the respiratory rate of the living being depending on the at least one variable.

15. The magnetic resonance imaging device of claim 14, further comprising at least one reception coil,
wherein the radio transmitter is signal-connected to the at least one reception coil and is configured to emit a message signal received by the at least one reception coil.

16. The magnetic resonance imaging device of claim 14, wherein the at least one variable comprises at least one frequency, at least one amplitude, at least one phase, or any combination thereof.

17. The magnetic resonance imaging device of claim 14, wherein the radio acquisition apparatus comprises at least one frequency filter.

18. The magnetic resonance imaging device of claim 16, wherein the radio acquisition apparatus comprises at least one frequency filter.

19. The magnetic resonance imaging device of claim 17, wherein the at least one frequency filter comprises a low-pass filter.

20. The magnetic resonance imaging device of claim 14, wherein the radio acquisition apparatus comprises at least one power detector, at least one frequency detector, at least one phase detector, or any combination thereof.
